(19) United States
(12) Patent Application Publication
(10) Pub. No.: US 2013/0039858 A1
(43) Pub. Date: Feb. 14, 2013

(54) NIR MATERIALS AND NANOMATERIALS FOR THERANOSTIC APPLICATIONS

(71) Applicants: Scott Chang Brown, Hockessin, DE (US); Amit Kumar Singh, Gainesville, FL (US); Parvesh Sharma, Gainesville, FL (US); Brij M. Moudgil, Gainesville, FL (US); Stephen R. Grobmyer, Gainesville, FL (US)

(72) Inventors: Scott Chang Brown, Hockessin, DE (US); Amit Kumar Singh, Gainesville, FL (US); Parvesh Sharma, Gainesville, FL (US); Brij M. Moudgil, Gainesville, FL (US); Stephen R. Grobmyer, Gainesville, FL (US)

(73) Assignee: University of Florida Research Foundation, Inc., Gainesville, FL (US)

(21) Appl. No.: 13/582,242

(22) PCT Filed: Feb. 24, 2011
(86) PCT No.: PCT/US2011/026044

§ 371 Date: Aug. 31, 2012

Related U.S. Application Data
(60) Provisional application No. 61/309,282, filed on Mar. 1, 2010.

(51) Int. Cl. C07D 487/22; A61K 49/22; A61K 49/10; G01N 21/64; C01B 33/18; A61K 49/00; A61K 49/04; B82Y 40/00; B82Y 15/00

(52) U.S. Cl. 424/9.3; 540/472; 424/9.1; 424/9.5; 424/9.6; 424/9.44; 436/172; 423/335; 977/773; 977/811; 977/920; 977/929; 977/930; 977/928; 977/896

(57) ABSTRACT

Novel fluorescent dye comprising metal oxide nanoparticles are prepared where the nanoparticles are as small as 3 nm or up to 7000 nm in diameter and where the dye is bound within the metal oxide matrix. In some embodiments the invention, novel dyes are covalently attached to the matrix and in other embodiments of the invention a dye is coordinate or ionic bound within the metal oxide matrix. A method for preparing the novel covalently bondable modified fluorescent dyes is presented. A method to prepare silica comprising nanoparticles that are 3 to 8 nm in diameter is presented. In some embodiments, the fluorescent dye comprising metal oxide nanoparticles are further decorated with functionality for use as multimodal in vitro or in vivo imaging agents. In other embodiments of the invention, the fluorescent dye comprising metal oxide nanoparticles provide therapeutic activity and incorporated therapeutic temperature monitoring.

### CROSS-REFERENCE TO RELATED APPLICATION

The present application claims the benefit of U.S. Provisional Application Ser. No. 61/309,282, filed Mar. 1, 2010, the disclosure of which is hereby incorporated by reference in its entirety, including any figures, tables, or drawings.

### BACKGROUND OF THE INVENTION

Fluorescence dyes are widely used for near-infrared imaging but many applications of these dyes are limited by disadvantageous properties in aqueous solution that include concentration-dependent aggregation, poor aqueous stability in vitro and low quantum yield. For example, a particularly useful dye, indocyanine green (ICG), is known to bind strongly to nonspecific plasma proteins, leading to rapid elimination from the body, having a half-life of only 3-4 min. Other limiting factors displayed by ICG include: rapid circulation kinetics; lack of target specificity; and optical properties of ICG that vary significantly due to influences such as concentration, solvent, pH, and temperature. To overcome some of these shortcomings the inclusion of the fluorescence dyes into micellar and nanoparticulate systems have been examined.

Attempts to encapsulate ICG into silica and polymer matrices have been met with only partial success. Much of this appears to stem from ICG's combined amphiphilic character and strong hydrophilicity, as it contains both lipophilic groups and hydrophilic groups that promote its concentration at interfaces and its interaction with the surfactants that are often necessitated in the particles synthesis, largely limiting its incorporation to the interior of nanoparticles. ICG displays a critical micelle concentration of about 0.32 mg/mL in H₂O and readily partitions into aqueous environments and ICG encapsulated in particulate matrices suffers from a leaching phenomena.

Yet encapsulated fluorescence dyes remain attractive for bio-imaging techniques that non-invasively measure biological functions, evaluate cellular and molecular events, and reveal the inner workings of a body. Fluorescent dye comprising nanoparticles are potentially useful for in vitro fluorescence microscopy and flow cytometry. Additionally, fluorescent dye comprising nanoparticles are potentially valuable for photo acoustic tomography (PAT), an emerging non-invasive in vivo imaging modality that uses a non-ionizing optical (pulsed laser) source to generate contrast, which is detected as an acoustic signal whose scattering is 2-3 orders of magnitude weaker than optical scattering in biological tissues, which is a primary limitation of optical imaging.

It is often necessary to use more than one imaging technique to integrate the strengths of each while overcoming the limitations of the individual techniques to improve diagnostics, preclinical research and therapeutic monitoring. Examples of other bio-imaging techniques include magnetic resonance imaging (MRI), positron emission tomography (PET), x-ray tomography, luminescence (optical imaging), and ultrasound. Typically, each of these techniques requires different contrast agents and using multiple bio-imaging techniques requires significantly greater time, expense and can impose diagnostic complications. If the fluorescent dye comprising nanoparticles include one or more additional contrast agents, multiple bio-imaging techniques could be carried rapidly or simultaneously. Multi-modal contrast bio-imaging agents are potentially important tools for developing and benchmarking experimental imaging technologies by carrying out parallel experiments of developing and proven techniques.

To these ends, effective and stable fluorescent dye comprising nanoparticles are needed. Methods of preparing these nanoparticles with a desirable size and composition are needed. Such novel nanoparticles could be employed for multiple biological applications, including imaging, even multiple bio-imaging techniques.

BRIEF SUMMARY OF THE INVENTION

Embodiments of the invention are directed to metal oxide nanoparticles having a near-IR (NIR) fluorescent dye bound to the metal oxide. The nanoparticles can have diameters of about 3 nm to about 7,000 nm, wherein one embodiment the diameters are less than 8 nm. The nanoparticles can be monodispersed in size distribution. The metal oxide can be silicon dioxide. The dye is bound within the metal oxide by one or more covalent bonds. The NIR fluorescent dye can be a naphthalocyanine or phthalocyanine metal complex. The nanoparticle can also include one or more of: a metal deposition, a moiety that provides luminescence, magnetic, or paramagnetic properties; or a moiety for x-ray opacity. The nanoparticle can include a habitat modifier bound to the metal oxide where the habitat modifier is an organic or inorganic group that alters polarity, pH, dielectric permittivity and/or porosity within the metal oxide matrix of the nanoparticle. The nanoparticle can include one or more optical limiting moiety such as a naphthalocyanine, phthalocyanine, fullerene, or functionalized fullerene. The nanoparticle can include a temperature indicating agent. The nanoparticle can include one or more chemotherapeutic agents, gene transfection agents, and/or gene silencing agents.

Another embodiment of the invention is a method to form metal oxide nanoparticles where the metal oxide is silica. The silica nanoparticles are formed by providing a mixture of at least one tetraalkoxysilane, an alcohol, water, and am ammonium catalyst and adding a polar aprotic solvent to yield a nanoparticle that has a diameter of about 3 to about 8 nm. To achieve the NIR fluorescent dye comprising nanoparticle above, a fluorescent dye that is covalently bound to a trialkoxysilane is included into the mixture. Alkytrialkoxysilanes or polyethylene glycol silane derivatives can also be included to modify the habitat within the silica nanoparticles.

Another embodiment of the invention is to administer these nanoparticles as a method of in vivo and in vitro imaging where a fluorescence signal can be detected. The nanoparticle can also allow the detection of luminescence, magnetic properties, paramagnetic properties, x-ray opacity; or any combination thereof Additionally the nanoparticles can include therapeutically active agents.

### BRIEF DESCRIPTION OF THE DRAWINGS

FIG. 1 is a plot of the mean size is given in terms of volume mean (MV) and number mean (MN) for silica nanoparticle prepared by a Stober synthesis with a solvent including the aprotic solvent DMF according to an embodiment of the invention.

FIG. 2 shows structures of IR-27, IR-1048, IR-1061, IR-775, IR-780, IR-783, IR-797, IR-806, and IR-820 that can form modified fluorescent dyes having metal oxide precursor groups by reactions such as those of Equations 1 and 2 according to an embodiment of the invention.

FIG. 3 show a TEM micrograph of 3-7nm NIR fluorescent dye comprising silica nanoparticles according to an embodiment of the invention.

FIG. 4 is a composite of fluorescence emission spectra of IR-820 comprising silica nanoparticles of various sizes according to an embodiment of the invention that were synthesized by the aprotic solvent modified Stober method using DMF as the aprotic solvent according to an embodiment of the invention where all nanoparticles were excited at 745nm.

FIG. 5 shows confocal image of live A549 cells with (left) and without (right) internalized 3nm NIR fluorescent dye comprising silica nanoparticles according to an embodiment of the invention that show as bright spots within the cells that have been stained with Hoescht and a green membrane stain.

FIG. 6 shows fluorescence emission spectra from IR-820 comprising silica nanoparticles according to an embodiment of the invention where irradiation is by a 5W laser for the time indicated from top to bottom.

FIG. 7 shows fluorescence emission spectra from prior-art ICG-doped silica nanoparticle irradiated by a 5W laser for the indicated time.

FIG. 8 shows a TEM micrograph of IR-820 comprising silica nanoparticles according to an embodiment of the invention prepared via a water-in-oil (cyclohexane/TX-100/hexanol) microemulsion synthesis using TEOS as the silica precursor where the scale bar=20nm.

FIG. 9 shows a TEM micrograph of IR-820 silane comprising silica nanoparticles according to an embodiment of the invention that are synthesized by a hydrothermal method using TEOS/CTAB/NaOH in water where the scale bar=50nm.

FIG. 10 shows a SEM micrograph of IR-780 silane comprising silica nanoparticles according to an embodiment of the invention using the Stober method from an Ethanol/TEOS/IR-780 modified silane mixture.

FIG. 11 shows NIR fluorescent images of various nanoparticle samples as indicted within the detailed description that were imaged using a Xenogen IVIS System with excitation at 745 nm and emission at A) 800 nm, B) 820nm, and C) 840nm.

FIG. 12 shows the structures of various exemplary naphthalocyanine and phthalocyanine complexed metals that can be condensed with metal oxide precursors to form NIR fluorescent dye comprising metal oxide nanoparticles according to embodiments of the invention.

FIG. 13 shows electron micrograph of silicon 2,3 napthalocyanine dihydroxide comprising silica nanoparticles according to an embodiment of the invention prepared by the Stober method where the top is an SEM image and the bottom is a TEM image.

FIG. 14 show an optical extinction profile for zinc naphthalocyanine comprising silica nanoparticles according to an embodiment of the invention.

FIG. 15 show an optical extinction profile for silicon 2,3 napthalocyanine dihydroxide comprising silica nanoparticles according to an embodiment of the invention.

FIG. 16 show an optical extinction profile for manganese (III) phthalocyanine chloride comprising silica nanoparticles according to an embodiment of the invention.

FIG. 17 shows a fluorescence spectrum of zinc naphthalocyanine comprising silica nanoparticles according to an embodiment of the invention upon excitation at 740 nm.

FIG. 18 shows a fluorescence spectrum of silicon 2,3 napthalocyanine dihydroxide comprising silica nanoparticles according to an embodiment of the invention upon excitation at 740 nm.

FIG. 19 shows optical images of A549 cells treated with silicon 2,3-napthalocyanine prior to irradiation and after irradiation with a 785nm Laser (500mW) for less than 2 seconds.

FIG. 20 shows Xenogen IVIS NIR fluorescent micrographs of IR-820 comprising silica nanoparticles according to an embodiment of the invention after subcutaneously injection in a nude mouse as indicated by the bright spot on right side of mouse and a second subcutaneously injection in the nude mouse on the left side with a prior art silica coated NIR quantum dots where the left image is for 800 nmn emission and the right image is for 820 nm emission.

FIG. 21 shows 745 excitation and 820 emission in Xenogen IVIS system of IR-820 comprising silica nanoparticles having gold speckles according to an embodiment of the invention after intratumoral injection where the insert in A) is the image of the mouse before injection, A) is the image 90 minutes post intratumoral injection, and B) is the image 24 hours post injection showing the translocation and accumulation of the nanoparticles, where the absence of autofluorescence from the mice organs enables easy detection of the nanoparticles.

FIG. 22 shows images of Balb/C mice inoculated with 4T1 luminescent tumor cells in the mammary fat pad of the mice.

FIG. 23 shows images of Balb/C mice inoculated with 4T1 luminescent tumor cells in the mammary fat pad of the mice after injection with of IR-780 and silicon 2,3 napthalocyanine dihydroxide comprising silica nanoparticles according to an embodiment of the after exposure to NIR light for combined photodynamic/photothermal therapy where the lack and decrease of luminescence indicates tumor destruction.

FIG. 24 shows images of Balb/C mice inoculated with 4T1 luminescent tumor cells in the mammary fat pad of mice after injection with saline solution.

### DETAILED DISCLOSURE OF THE INVENTION

Embodiments of the invention are directed to the preparation of metal oxide comprising nanoparticles. These metal oxide nanoparticles can range from about 3 to about 7,000 nm. Some embodiments of the invention are directed to a method of preparing metal oxide comprising nanoparticles less than 8 nm in cross section (diameter for an effectively spherical particle) with a narrow size distribution (nearly monodispersed) having a mean size with nearly the same volume percent (MV) and number percent (MN). Some embodiments of the invention are directed to metal oxide nanoparticles that further comprise fluorescent dyes, which are referred to as fluorescent dye comprising nanoparticles herein. The fluorescent dyes include near-IR (NIR) and visible dyes functionalized to be covalently bound within and/or upon the nanoparticle. Some embodiments of the invention are directed to methods of preparing modified fluorescent dyes, and a method of preparing fluorescent dye comprising nanoparticles by inclusion of the modified fluorescent dyes in a reaction mixture with metal oxide precursors. Other embodiments of the invention are directed to multimodal fluorescent dye comprising nanoparticles, where at least one other component is included in the nanoparticle such that a plurality of independent properties are displayed by the nanoparticles, which can be sequentially or simultaneously exploited for targeting, imaging, therapeutic, or other activities.

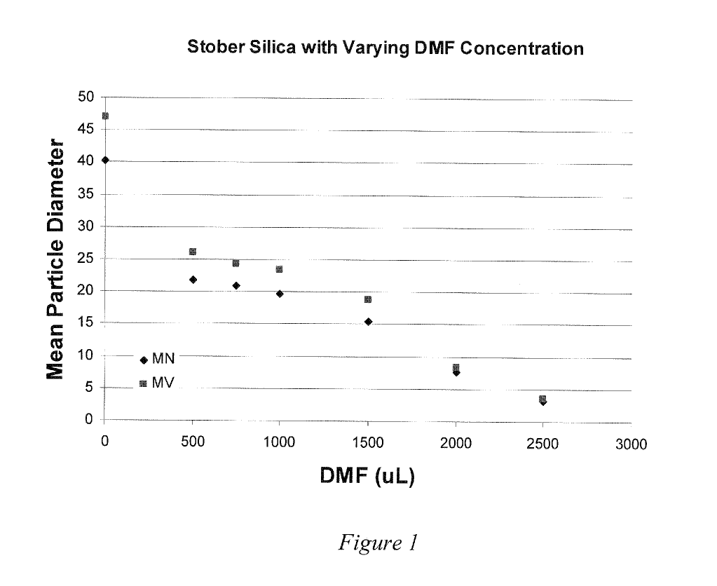

Metal oxide comprising nanoparticles can be prepared by microemulsion routes, Stober synthesis protocols and via modified mesoporous silica synthesis routes. In an embodiment of the invention, a modified Stober synthesis involves the condensation of at least one metal oxide precursor in the presences of at least one alcohol and at least one polar aprotic solvent. The resulting metal oxide nanoparticle can include silicon dioxide, titanium dioxide, cerium oxide, aluminum oxide, and zinc oxide. In one embodiment of the invention, a method of metal oxide nanoparticle synthesis involves the combination of the metal oxide precursor, for example an alkoxy substituted metal, for example tetraethoxysilane (TEOS) or tetramethoxysilane (TMOS), is combined with an alcohol, for example ethanol or methanol, ammonia or a basic ammonium salt, and a polar aprotic solvent, for example dimethylformamide (DMF), dimethylsulfoxide (DMSO), acetonitrile (MeCN), tetrahydrofuran (THF), 1,4-dioxane, and acetone with or without agitation. After a sufficient period of time, for example 12 hours, the metal oxide precursor is converted to metal oxide nanoparticles with a cross section (diameter of a spherical nanoparticle) of about 3 to about 7 nm, depending upon the proportion of polar aprotic solvent used, where the greater the proportion of polar aprotic solvent, the smaller the cross section of the nanoparticle. This dependence is illustrated in FIG. 1 where a plot of the mean particle size of a silica nanoparticle is shown to decrease with increased DMF volume for a Stober synthesis using otherwise identical volumes of TEOS, ethanol, and ammonia. As can also be seen in FIG. 1, the mean based on volume percent (MV) and mean based on number percent (MN) are nearly identical for nanoparticles less than 10 nm in size. As can be seen in FIG. 1, in the absence of the polar aprotic solvent the particle diameter is larger than 40 nm. Consistent preparation of silica nanoparticles smaller than 8 nm are not possible by the traditional Stober as the initial nucleated silica nanoparticles display a radius of gyration that is about 4 nm (about 8 nm in diameter) using TMOS in methanol and about 8 nm (about 16 nm in diameter) using TEOS in ethanol, as disclosed in D. L. Green et al., Journal of Colloid and Interface Science 2003, 266, 346-58.

In other embodiments of the invention fluorescent dye comprising nanoparticles can be formed by inclusion of a modified fluorescent dyes with metal oxide precursors and carrying out nanoparticle synthesis by a microemulsion route, a modified mesoporous silica synthesis route, a Stober synthesis, or the modified Stober synthesis according to an embodiment of the invention. The fluorescent dye can be a NIR fluorescent dye, which can display emission from about 750 nm to about 820 nm that can be modified to include a group that can be co-condensed with the metal oxide precursor to become a constituent of the metal oxide comprising nanoparticle, a fluorescent dye comprising nanoparticle. For example, in embodiments of the invention where the metal oxide is silicon oxide, the modified fluorescent dyes comprise NIR-dye conjugates having a silane terminus such that the silica forming synthesis allows preparation of the fluorescent dye comprising nanoparticle without separation of unincorporated dye conjugate from the final product as the modified fluorescent dye is bound within the metal oxide (silica) comprising nanoparticle.

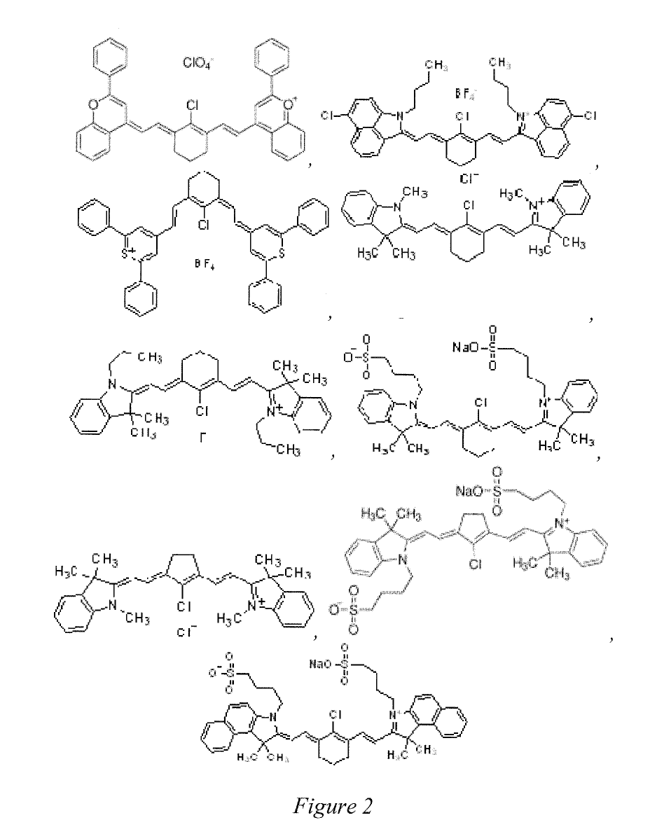

In embodiments of the invention, the NIR-dye can comprise a conjugated system that is bound to a trialkoxysilane through a series of 3 to 20 carbon-carbon bonds that can be uninterrupted or interrupted by a O, S, NH, NR, C(O)O, C(O)NH, C(O)NR. In embodiments of the invention the conjugated unit is derived from an NIR-dye that contains a reactive halide, for example a chloride, bromide or iodide, or its equivalent, for example an arysulfonate, that can act as a leaving group. The structures of commercially available NIR-dyes that can be used for modified fluorescent dyes, according to embodiments of the invention, are shown in FIG. 2, which include IR-820, IR-780, IR-1048, IR-1061, IR-27, IR-775, IR-783, IR-797, and IR-806. As can be followed in the exemplary synthesis indicated by Equations 1 and 2, below, the dye is coupled with a reactive silane, for example a trialkoxysilane, where a linking unit is included between the dye portion and the condensable silane group of the modified fluorescent dye. The linking unit can be a 3 to 20 carbon alkyl chain that can be uninterrupted or interrupted by a O, S, NH, NR, C(O)O, C(O)NH, C(O)NR, aromatic or other group which may be formed to couple the reactive silane to the dye portion of the modified fluorescent dye and where R is, for example, a 1 to 3 carbon alkyl group.

The modified fluorescent dye can be formed by nucleophilic substitution at the site of the reactive halide or equivalent with the reactive halide or equivalent being displaced by a nucleophile attached to the linking group. The nucleophile can be an N, O, S, or C atom and can be in a neutral or anionic state. For example the nucleophile can be the nitrogen of an amine. The nucleophilic substitution reaction can be carried out in the presence of a catalyst and/or a promoter or in the absence thereof. The nucleophilic substitution can be carried out with a nucleophile containing linking unit that can be attached to the silane, another metal oxide precursor, or an alternate functional group by which the silane or another metal oxide precursor can be attached by a subsequent reaction. The subsequent reaction can be any condensation, addition, or exchange reaction, for example the reaction can be a condensation of a carboxylic acid or its active ester with an amine containing silane, for example an aminopropylsilane, to form a interrupting amide (C(O)NH) unit in the linking unit and connect the silane to the dye. As needed, any intermediate structure or the final modified fluorescent dye is purified by any appropriate technique, such as extraction, crystallization, or chromatography as can be appreciated by one skilled in the art.

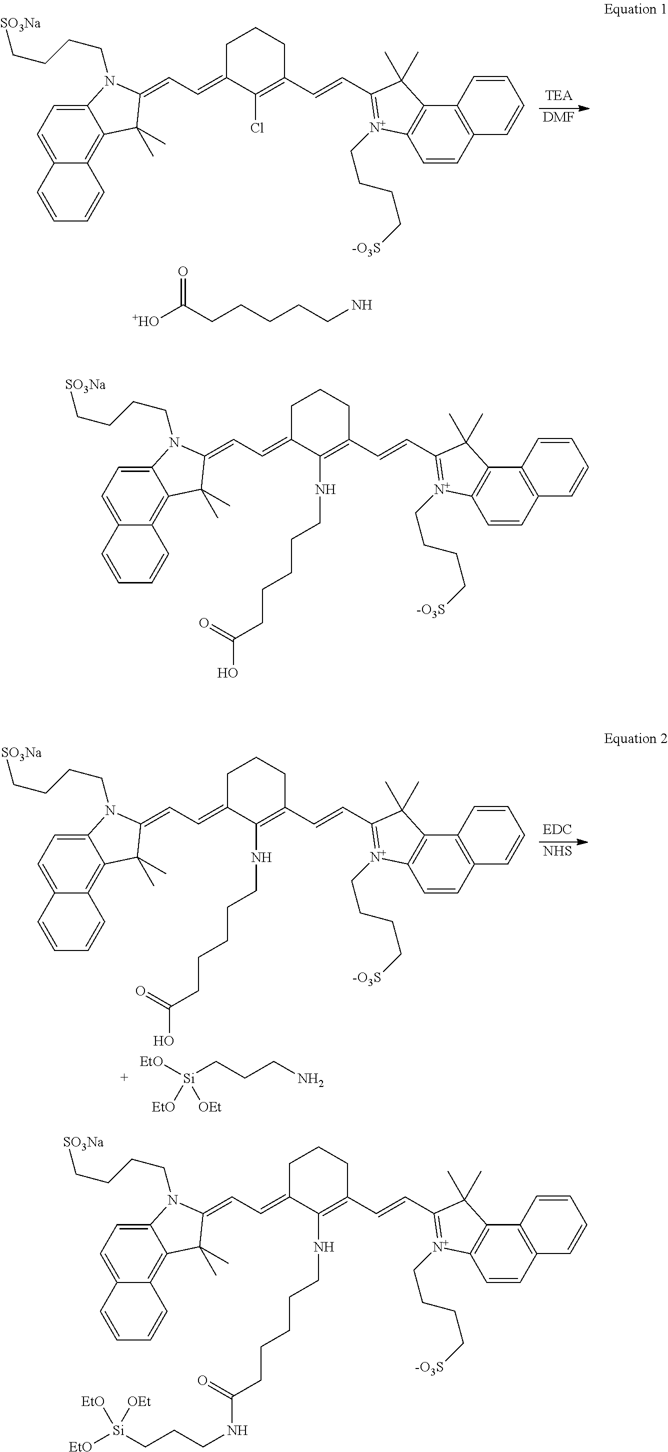

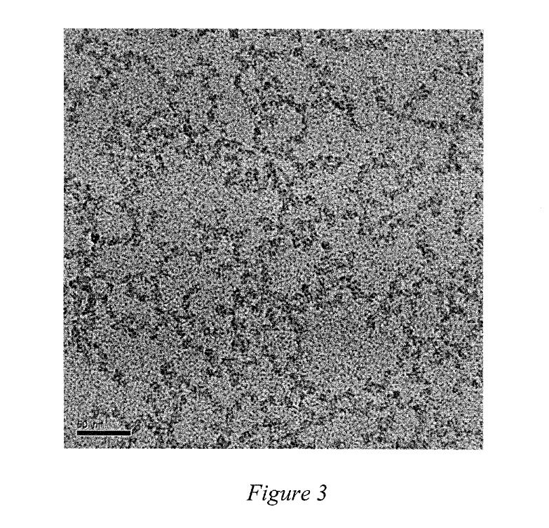

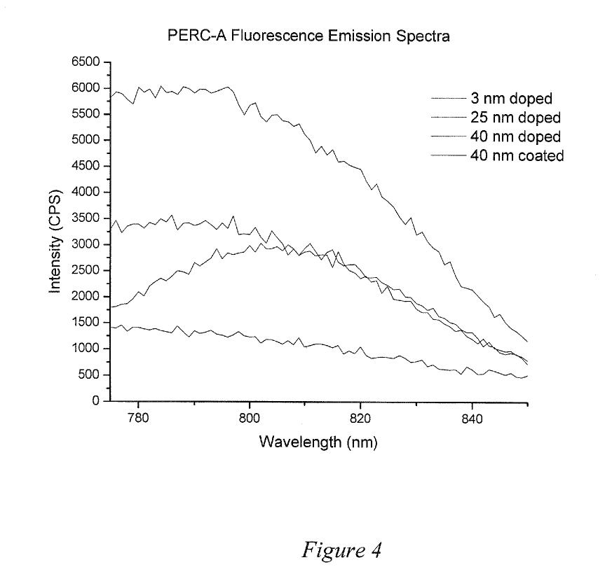

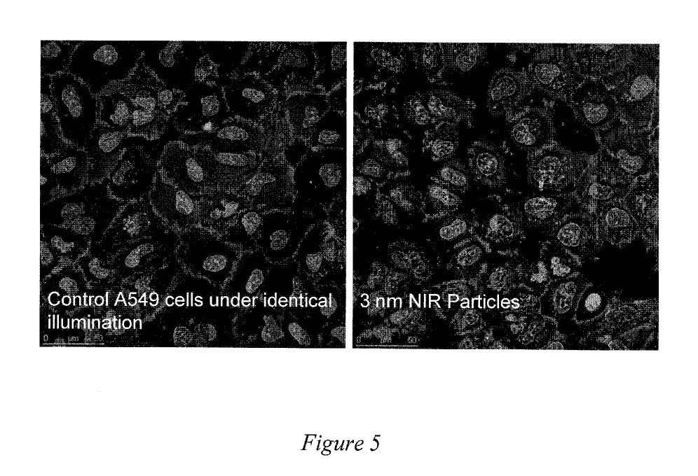

The modified fluorescent dyes can be incorporated into and/or onto the metal oxide comprising nanoparticle by any of the methods given above. For example, the modified fluorescent dye can contain a trialkoxysilane group and be co-condensed with tetraalkoxysilanes by the modified Stober synthesis according to an embodiment of the invention. In this manner, fluorescent dye comprising nanoparticles of less than 8 nm can be prepared, as illustrated in. FIG. 3 for fluorescent dye comprising silica nanoparticles that are 3 to 7 nm in diameter. These small fluorescent nanoparticles can display a fluorescent shift to longer wavelengths relative to larger nanoparticles of equivalent composition, as illustrated in FIG. 4. These very small nanoparticles can penetrate cell walls as illustrated for A549 lung carcinoma cells that were incubated with 3 nm fluorescent dye comprising silica nanoparticles as shown in FIG. 5. Alternately, according to other embodiments of the invention, larger fluorescent dye comprising nanoparticles can be formed by alternate synthesis of metal oxide nanoparticles, such as a Stober synthesis, as indicated at zero DMF concentration of FIG. 1, a microemulsion route, or a modified mesoporous silica synthesis route. In other embodiments of the invention, fluorescent dye comprising nanoparticles can be formed by having mesoporous silica or other metal oxide treated with the modified fluorescent dyes, where the modified fluorescent dyes act as coupling agents to condense onto the surfaces within the pores and on the external surface of the mesoporous silica or other metal oxide.

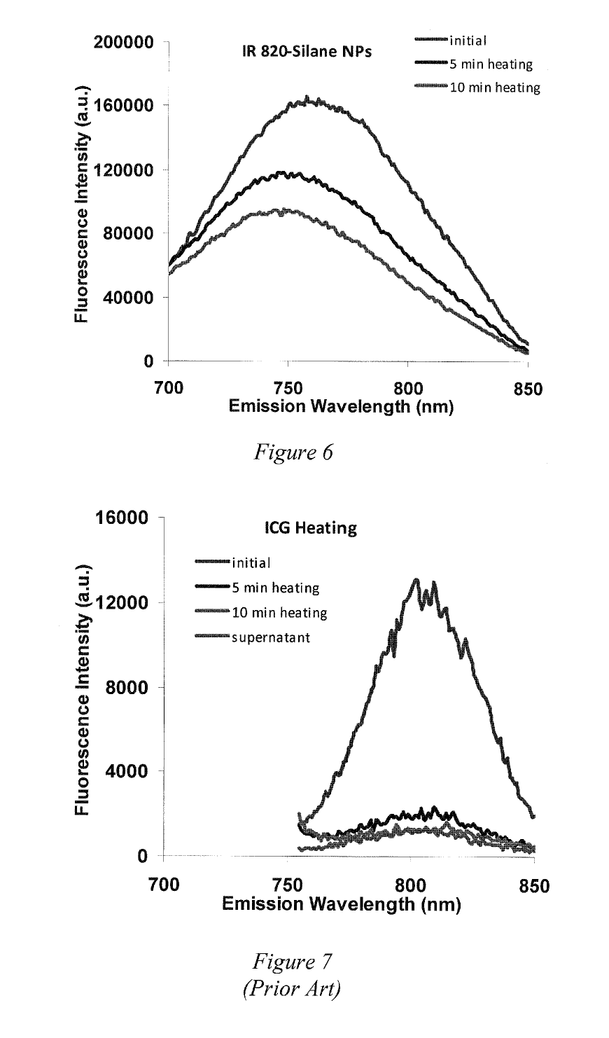

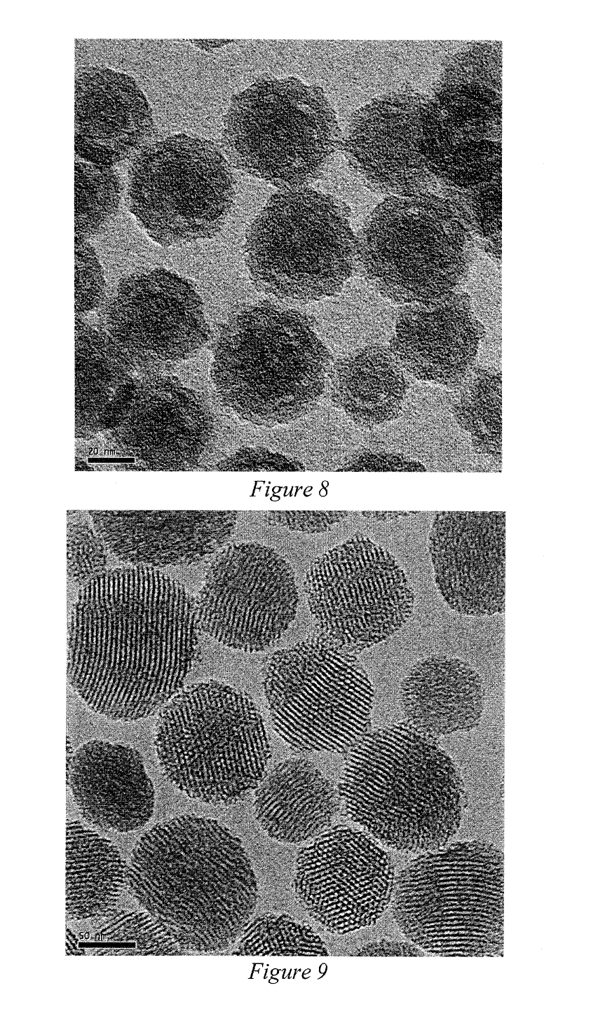

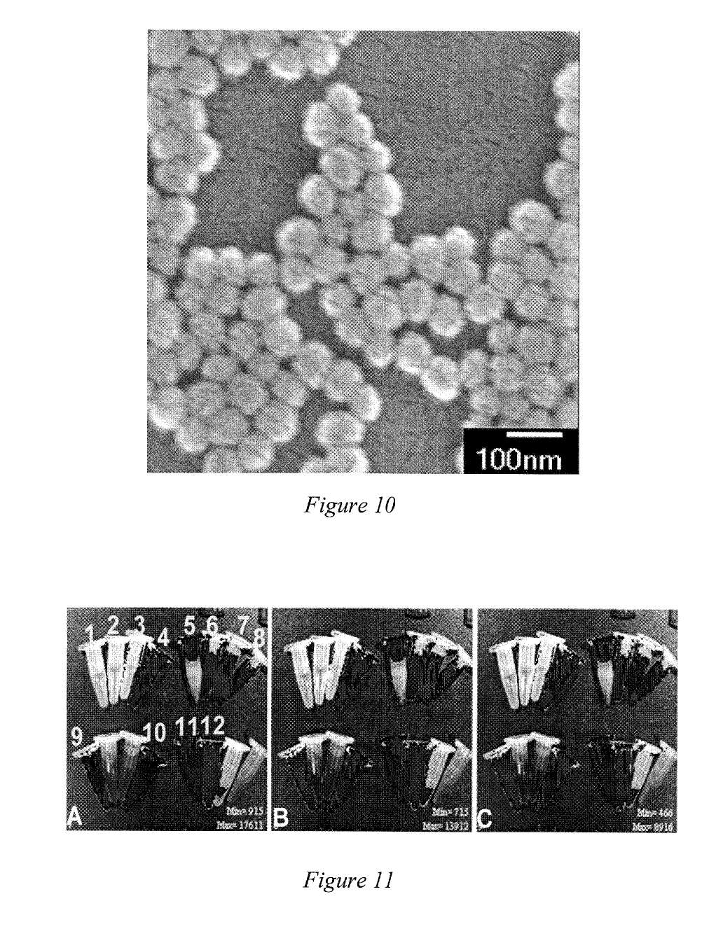

The fluorescent dye comprising metal oxide nanoparticles, according to an embodiment of the invention, display high stability to photo bleaching than do prior art NIR dye comprising nanoparticles that do not have a covalently bound group that is capable of condensing with the metal oxide precursors. FIGS. 6 and 7 show the decrease in fluorescence with irradiation time for IR-820-silane comprising nanoparticles according to an embodiment of the invention and prior art ICG-doped silica nanoparticles, respectively, that are irradiated with a 5W laser for 5 and 10 minutes. As can be seen in FIG. 6, the IR-820-silane comprising nanoparticles retain about 50% of their emission intensity after 10 minutes of irradiation, while that of the prior art ICG-doped silica nanoparticles have lost nearly all of their emission intensity after 5 minutes. FIGS. 8 and 9 show IR-820-silane comprising nanoparticles that were prepared by a microemulsion route and a modified mesoporous silica synthesis, respectively, and FIG. 10 shows IR-780-silane comprising nanoparticles prepared by a Stober synthesis. Fluorescent dye comprising nanoparticles show stable fluorescence emission, FIG. 11 shows emission spectra at a) 800 nm, b) 820 nm and c) 840 nm for 745 nm excited vials containing various control and fluorescent dye comprising nanoparticles. Vial 1 contains silica nanoparticles synthesized in the presence of free IR-820 dye. Vial 2 contains silica nanoparticles synthesized with the silane free IR-820 aminocaproic acid intermediate with a silica condensation is catalyzed by ammonium hydroxide. Vial 3 contains silica nanoparticles synthesized with IR-820 aminocaproic acid intermediate and APTS without condensation where the silica condensation was catalyzed by ammonia. Vial 4 contains silica nanoparticles synthesized with a silane modified IR-820 according to an embodiment of the invention with 100 μL of unpurified dye where the silica condensation was catalyzed by ammonia for an EDC/NHS reaction over 2 hours. Vial 5 differs from vial 4 by the incorporation of 200 μL of unpurified dye. Vials 6 and 7 differs from vial 4 in that the condensation was carried out in an AOT microemulsion to yield 15 nm particles and 20 nm particles, respectively. Vial 8 differs from vial 4 in that IR-780 rather than IR-820 is in the silane modified dye. Vial 9 differs from vial 4 in that ammonia carbonate was used as the condensation catalyst. Vial 10 contains silica particles that are like those of vial 5 but with gadolinium is also incorporated into the nanoparticle by a silane chelate (N-(Trimethoxysilyl-propyl)ethyldiamine triacetic acid trisodium salt). Vial 11 differs from vial 5 in that ammonia carbonate was used as the condensation catalyst. Vial 12 has identical contents to vial 4 that had been aged for 3 months in the absence of light in water at room temperature. From these results, it is clear that the effective incorporation of IR-820 into a silica nanoparticle is dramatically enhanced by the covalent attachment of a group that can be condensed with the tetraalkoxysilanes.

In an embodiment of the invention, to improve the photostability and luminescent properties of the particulate materials additional molecules and metal oxide precursor derivatives may be incorporated within the particle matrix as habitat modifiers. For instance, the incorporation of a 12-carbon alkyl silane in the Stober synthesis of IR-780-silane NIR fluorescent particles results in an order of magnitude increase in quantum yield. Habitat modifiers are molecules that are included to alter the local polarity, pH, dielectric permittivity, and/or porosity, of the internal particle structure. Examples of habitat modifiers include polyethylene glycol silanes, alkyl silanes, and other polymer-silane derivatives.

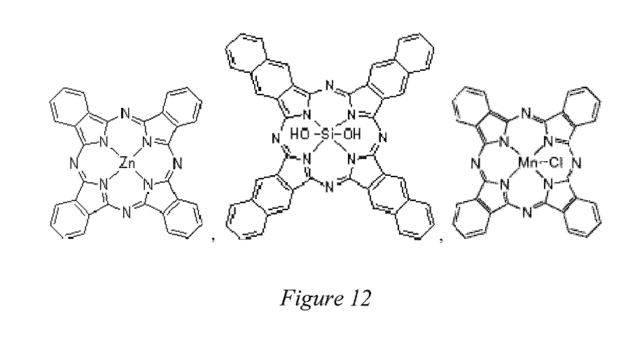

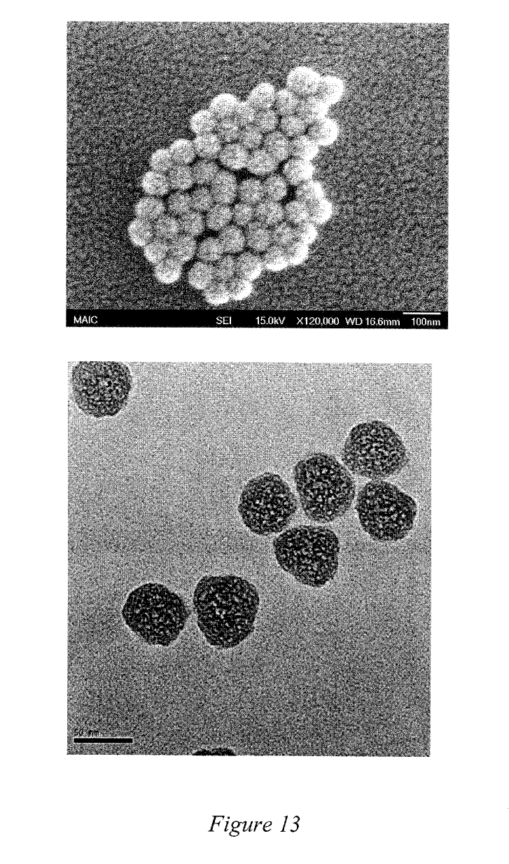

In other embodiments of the invention the particles described above are additionally doped with optical limiting moieties such as naphthalocyanine and/or phthalocyanine materials for therapeutic applications as well as imaging applications. Metal, metal oxide, polymer or hybrid nanoparticles may be doped with naphthalocyanine and/or phthalocyanine materials for both therapeutic and imaging applications. Metal containing and metal free naphthalocyanine and phthalocyanine complexes, for example, those illustrated in FIG. 12, can be incorporated into nanoparticles, for example by a Stober synthesis of tetraalkoxysilanes to form the novel therapeutic and imaging agents. In other embodiments of the invention, one or more modified fluorescent dyes, for example the dye formed according to Equation 2 above, can be included with one or more metal containing phtalocyanine complex. Any metal, for example, as illustrated herein by Si, Zn or Mn, can be incorporated into the fluorescent nanoparticles, as indicated by Table 1 below. In general, superior incorporation of the phthalocyanine occurs with a metal that can form a covalent, coordinate, or ionic bond to an oxygen within the metal oxide matrix, although, in some embodiments, the phthalocyanine metal complex can be incorporated within the matrix without any specific interactions to the matrix. FIG. 13 shows the SEM and TEM images of silica particles of about 50 nm in diameter that incorporate silicon 2,3 napthalocyanine dihydroxide by a Stober synthesis. In some applications using these nanoparticles according to embodiments of the invention, the phthalocyanine complex is disseminated from the fluorescent nanoparticles.

TABLE 1
Encapsulation of phtalocyanine dyes into silica nanoparticles

| | Percent | Approximate % |
|---|---|---|
| Dye | Yield | Encapsulation |
| Zinc naphthalocyanine | 83.9 | 100 |
| Silicon 2,3 napthalocyanine dihydroxide | 89.9 | 100 |
| Manganese (III) phthalocyanine chloride | 83.9 | 50 |

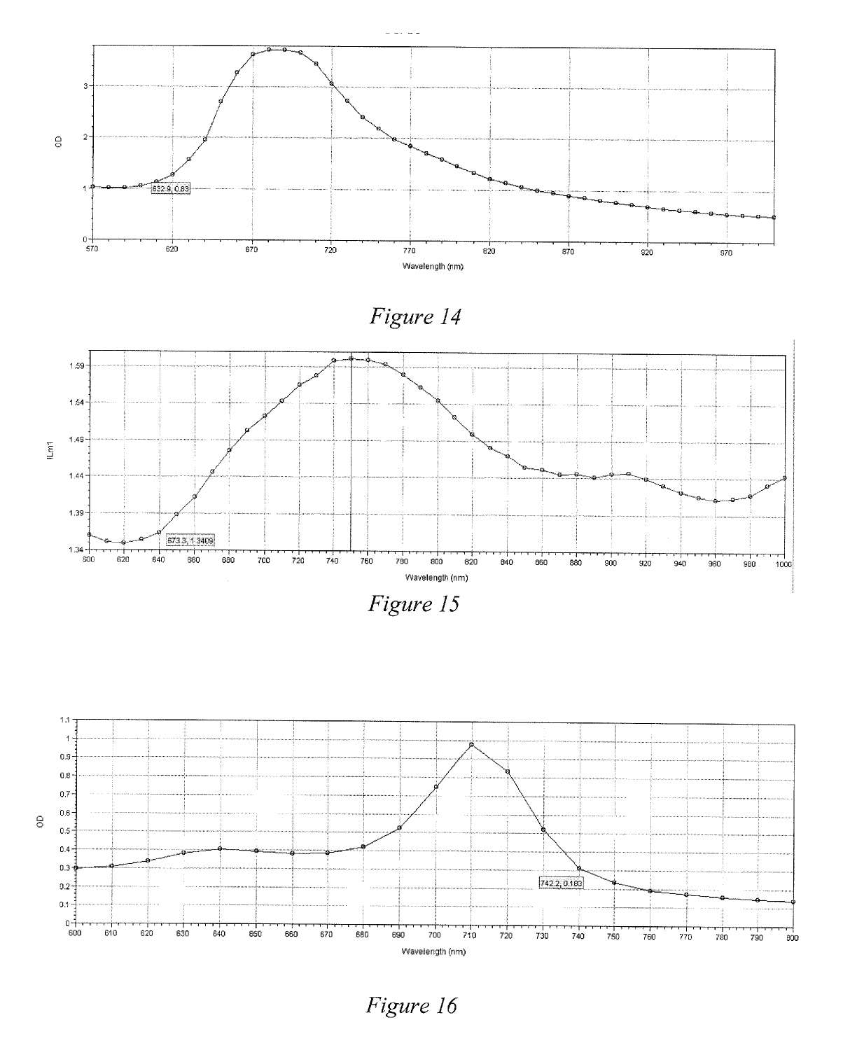

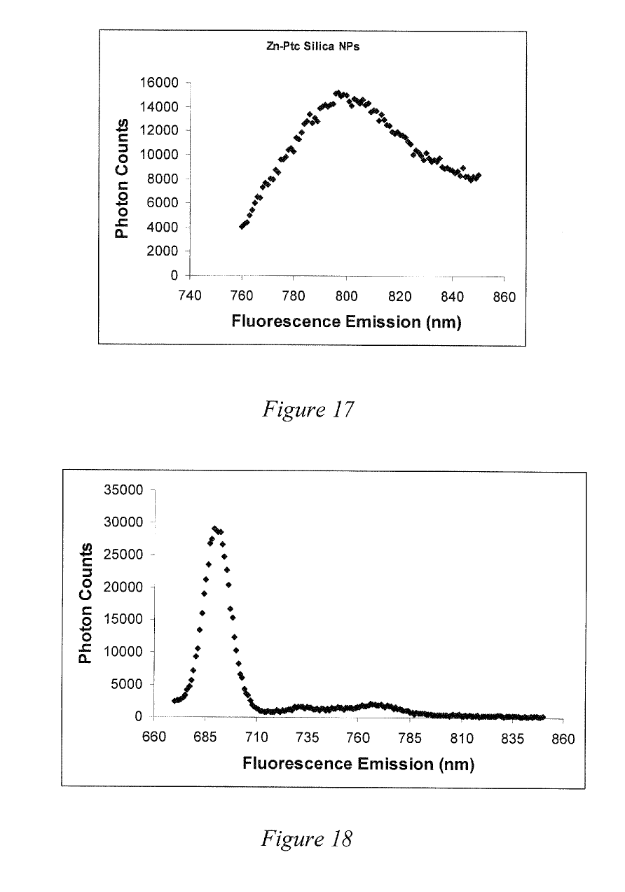

As shown in FIGS. 14-16, silica particles containing the dyes of Table 1 display optical extinction profiles with maximums in the NIR, indicative of dye incorporation, and fluorescence spectroscopy, as shown in FIGS. 17 and 18 confirm the presence of the dye and their capability to perform fluorescence imaging.

The novel phtalocyanine comprising metal oxide nanoparticle can be used for phototherapy according to an embodiment of the invention. For example, Human Aveolar Type II adenocarcinoma cells (A549, ATCC Manassass, Va.) were incubated with the phthalocyanine dye doped silica particles of Table 1 in RPMI 1640 media with 1% serum for 40 hours and subsequently irradiated with a 785nm Laser (500mW) for less than 2 seconds. Cytotoxicity of the nanoparticles without irradiation was determined by LDH release using an. LDH kit (Roche), results of which are summarized in Table 2, where none of the samples exhibited appreciable toxicity above a control.

TABLE 2
Cytotoxicity of formulated NIR dye doped silica
nanoparticles to Human A549 cells.

| | | | Si-2,3- | | | |
|---|---|---|---|---|---|---|
| | Zn | | napthalocyanine | | Mn(III) | |
| Dye | naphthalocyanine | | (OH)₂ | | phthalocyanine Cl | |
| μg/mL | 50 | 500 | 50 | 500 | 50 | 500 |
| % | 0.2 | 5.50 | 0 | 0.70 | 0.40 | 0 |
| cytotoxicity | | | | | | |

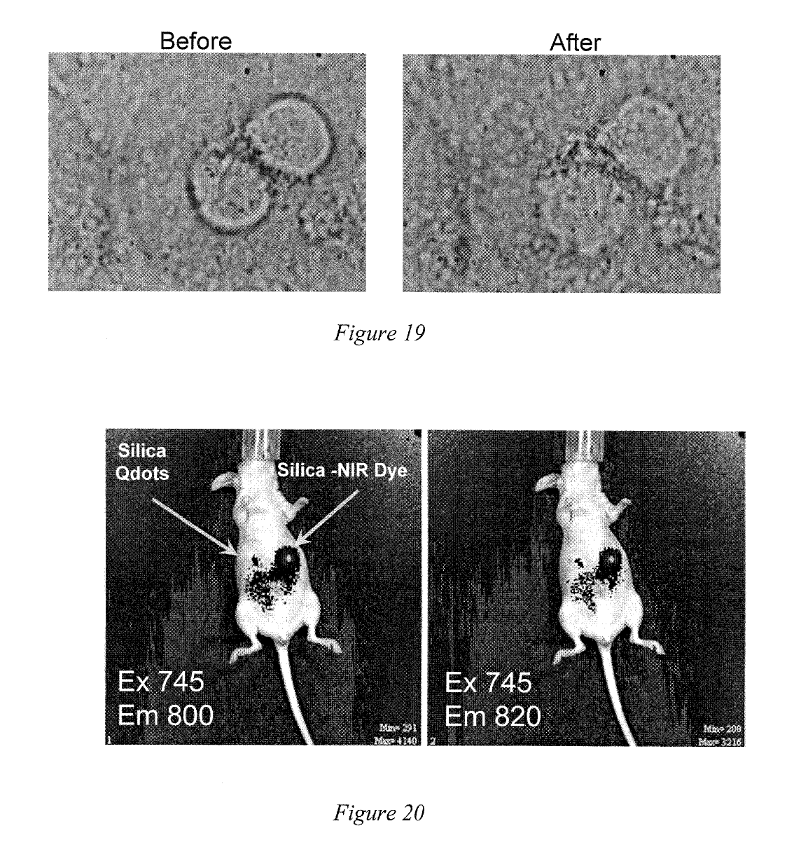

FIG. 19 presents cells prior and after exposure to NIR light for less than 2 seconds using the Renishaw Invia Raman laser. After exposure to NIR light the cells containing the NIR dye doped particles were destroyed, and cell death was confirmed by trypan blue dye uptake (not shown).

The fluorescent dye comprising nanoparticles according to embodiments of the invention can be used for in vivo imaging. FIG. 20 shows the image generated from 50 nm IR-820-silane comprising silica nanoparticles after subcutaneously injected into a mouse using a Xenogen IVIS system. FIG. 20 also shows, for comparison the fluorescence of silica coated q-dots of the same mass which were prepared from a commercial Invitrogen product. The dye comprising nanoparticles, according to embodiments of the invention, are significantly higher in intensity than that of the silica coated q-dots.

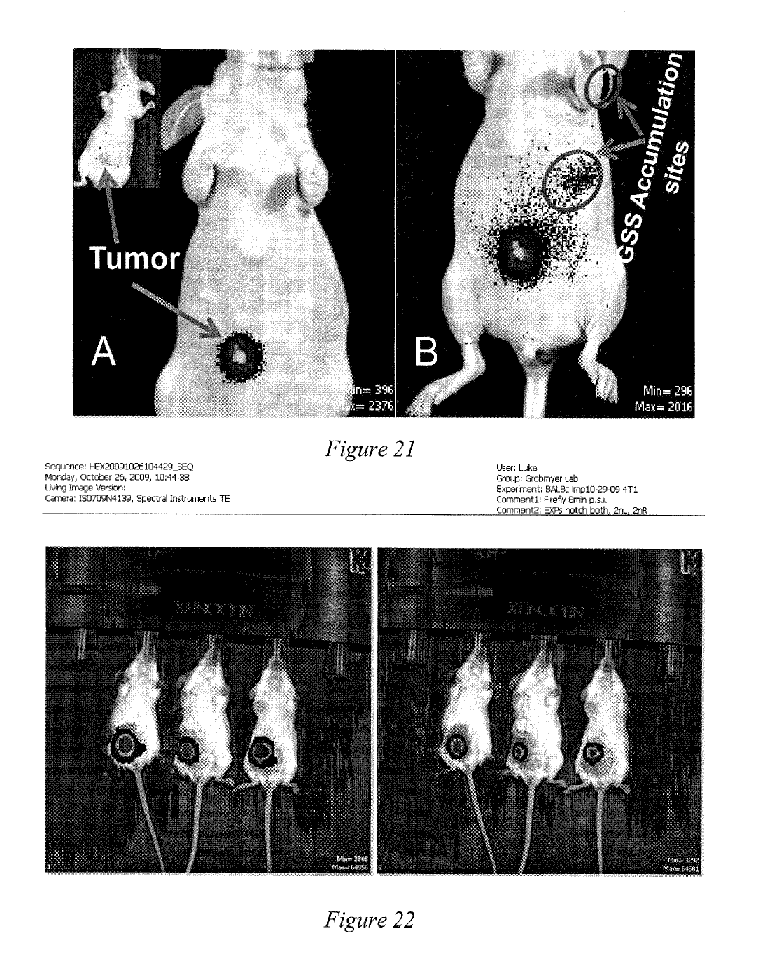

In other embodiments of the invention, the fluorescent dye comprising nanoparticles are further decorated with one or more additional groups and/or structures that impart one or more additional activities to the fluorescent dye comprising nanoparticles, multimodal fluorescent dye comprising nanoparticles, that allow the nanoparticles to selectively segregate to (target) a particular site, for example tumor cells, to permit detection by at least one other additional non-fluorescence technique, or to deliver or act as a therapeutic for treatment of the target. Preparation of the multimodal fluorescent dye comprising nanoparticles can be carried out by decoration of the fluorescent dye comprising nanoparticles of the present invention with a metal, such as a gold speckle, as an x-ray contrasting agent and/or a transition metal chelate or lanthanide chelate, such as Mn-EDTA (ethylene diamine tetraacetic acid) or Gd-DTPA (diethylene triamine pentaacetic acid), as a MRI contrasting agent bound to the surface of the nanoparticle in the manner taught in Sharma et al., International Application No. PCT/US2008/74630; filed Aug. 28, 2008, and incorporated herein by reference, wherein it teaches a fluorescent dye containing silica nanoparticle where the novel modified fluorescent dyes are substituted for the flouroscein isothiocyanate (FITC) of the relatively large nanoparticles formed in a reverse micelle taught therein. Additionally, the fluorescent dye comprising nanoparticles are coated with an additional metal oxide barrier coating to separate the fluorescent dye group from any metal that can otherwise quench the dye. Alternately, iron oxide can be incorporated into the fluorescent dye comprising nanoparticles to form multimodal fluorescent dye comprising nanoparticles where the iron oxide is used in addition to or in place of any transition metal chelate or lanthanide chelate to enhance MRI contrast. The ability to carry out in vivo imaging with multimodal fluorescent dye comprising nanoparticles is shown in FIG. 21 where IR-820 comprising silica particles are rendered gold speckled, as disclosed in Sharma et al. where a silica barrier coating was placed between the dye-containing core and the gold-speckled to avoid or reduce any dye quenching upon deposition of the gold. The gold-speckled-silica nanoparticles (GSS) that had been intratumorally injected into a tumor-bearing nude mouse displayed a significant fluorescence signal that can be followed over 24 hours or more for the translocation of the GSS nanoparticles when imaged using a Xenogen IVIS system.

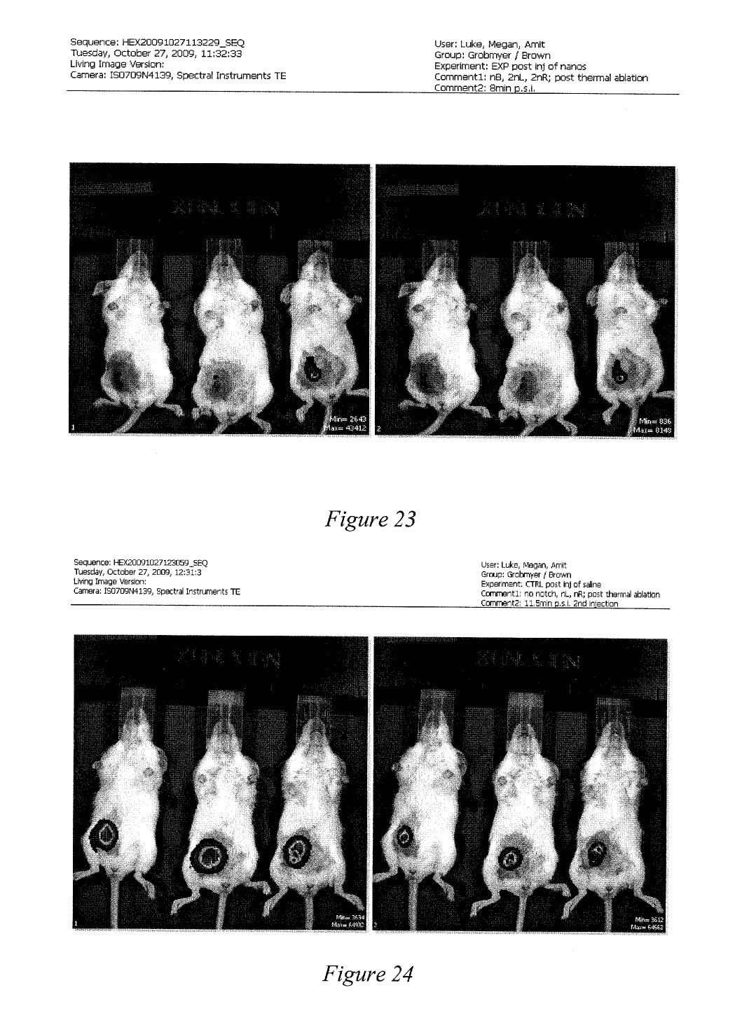

The fluorescent dye comprising metal oxide nanoparticles, according to embodiments of the invention, can be used for tumor ablation. For example, Balb/C mice were inoculated with 4T1 luminescent tumor cells in the mammary fat pad and tumors develop over one week, displaying bioluminescence that corresponds to the presence of 4TI cancer cells as shown in FIG. 22. Subsequently, 50 μL of a 1 mg/mL suspension of IR-780 silane/silicon 2.3 naphthalocyanine comprising silica nanoparticles were injected into the orthotopic tumors and exposed to NIR light for combined photodynamic/photothermal therapy. The decrease in bioluminescence indicates that a significant portion of the tumor have been destroyed in each mouse, as shown in FIG. 23, as opposed to that of control mice that were injected with saline and not the IR-780 silane/silicon 2.3 naphthalocyanine comprising silica nanoparticles, where little, if any, decrease in the luminescence was observed, as shown in FIG. 24.

The fluorescent dye comprising nanoparticles or multimodal fluorescent dye comprising nanoparticles can be used for theranostic (simultaneously therapeutic and diagnostic) agents according to embodiments of the invention. For example, these nanoparticles can be intratumorally injected into a tumor-bearing nude mouse and subsequently irradiated using a NIR laser, for example using a Xenogen IVIS system, to significantly elevate the temperature at the site of the tumor. Theranostic NIR and MRI active multimodal fluorescent dye comprising nanoparticles according to embodiments of the invention can be modified biologically-targeting groups where the injected nanoparticles can be used to treat and monitor the effectiveness of the treatment of a mammalian patient. In an embodiment of the invention, a built in therapeutic temperature relaying systems can further enhance thermal/dynamic ablation therapies by providing feedback on its effectiveness. Some tumors can occur in a location that influences the ability of the theranostic multimodal fluorescent dye comprising nanoparticles to be sufficiently heated to the required therapeutic temperature, for example, when an adjacent vasculature acts as a heat sink, or at a depth or otherwise shielded position that results in poor penetration of the necessary electromagenetic waves. In such situations, irradiation of the nanoparticles can provide feedback that the required level of heat has been achieved. In one embodiment of the invention, an additional NIR fluorescent dye or an MRI active chelate is bound to the metal oxide matrix by a linker that is temperature sensitive. The linker is susceptible to rapid thermal degradation. A temperature indicating agent, such as a dye and/or chelate, is bound via a thermal degradable linker that inhibits molecular leaching at nominal body temperatures but allows rapid release of the indicating agent once the desired therapeutic temperature is reached by the nanoparticle when the linker is cleaved. The degradable group can be a covalent (allowing, for example, radical formation or retro-addition reactions), ionic, coordinate or electrostatic based linkage. When this linker is cleaved, the diffusion of the dye or chelate can either generate a new signal or diminish an existing signal from the multimodal fluorescent dye comprising nanoparticles to communicate that the desired temperature has been achieved. Alternately, the nanoparticles can include a diffusible quenching agent and/or a water exchange limiting molecule that are physically fixed to or within the nanoparticle until the desired therapeutic temperature is achieved upon which it can diffuse from the nanoparticle. In the former case, the quenching agent can inhibit a signal by a dye until the therapeutic temperature is achieved or can be a physically attached dye that provides a signal but is released from the nanoparticle to result in rapid signal loss after temperature induced physical changes to the nanoparticle occur. Diffusible temperature indicating agents can include, for example indocyanine green (ICG), IR-820 derivatives, IR-780 derivatives, gadolinium 1,4,7,10-tetraazacyclododecane-1,4,7,10-tetraacetic acid (Gd-DOTA), and Gd-DOTA-polylysine. Quenchers and water exchange limiting group such as a hydrophobic molecule.

Information derived using this technology allows for a timely decision of subsequent treatments, often alternate treatments when it is determined that the required temperature has not been achieved. Use of these theranostic multimodal fluorescent dye comprising nanoparticles permit an initial inexpensive noninvasive treatment to be tried prior to an operation or use of more complicated and expensive treatment routes, such as surgical resection. For example, a deep breast tumor could be given NIR light treatment in a manner that the physician could determine if therapeutic levels of heat were generated using a portable NIR optical mammography device. If sufficient temperature had not been achieved, the patient could undergo non-invasive radiofrequency therapy that is monitored by MRI using the theranostic multimodal fluorescent dye comprising nanoparticles.

In another embodiment of the invention, drugs and/or gene silencing or transfection agents may be incorporated into the multimodal fluorescent dye comprising nanoparticle. As with the theranostic nanoparticles of above, upon laser illumination, the additional therapeutic agents can be selectively eluted for site specific therapy. The additional therapeutic agents can be included with temperature indicating agents that signal release.

Following are examples which illustrate procedures for practicing the invention. These examples should not be construed as limiting.

### MATERIALS AND METHODS

A standard Stober synthesis was repeated five times where 0.38 mL TEOS was added to 11.4 mL of ethanol in each if seven vials, followed by addition of 0.57 mL of ammonia to each vial. Subsequently, an aliquot of DMF was added to six of the vials and all of the vials were capped and the contents stirred. The quantity of DMF added varied, where specifically, 0.50, 0.75, 1.00, 1.50, 2.00, and 2.50 mL of DMF was added to individual vials. After 12 hours, particle size was measured by dynamic light scattering (Microtrac Nanotrac). Particle yield was determined by a residue analysis where a known weight of the suspensions in weighing pans was dried overnight in an oven and reweighed. Regardless of DMF content, no difference in the mass yield of particles was observed, although the size of the synthesized particles almost linearly decreased with increasing DMF content, as indicated in FIG. 1. Furthermore, the mean number and mean volume values were also measured and, as indicated in FIG. 1, the polydispersity of the particles decreases as the size decreases.

The modified IR-820-silane fluorescent dye produced according to Equation 2, above, and used to prepare the above IR-820-silane fluorescent dye comprising silicon oxide nanoparticles, was synthesized in the following manner. IR-820, 300 mg, was dissolved in DMF to yield an approximate concentration of 30 mg/mL. The dye was mixed with 130 mg of 6-aminocaproic acid with about 200 μL of the catalyst triethylamine and heated to 85 ° C. for 3 hours under a nitrogen atmosphere to form the amine substituted product of Equation 1, which was subsequently mixed with 3-aminopropyltriethoxysilane (APTS) and 1-Ethyl-3-[3-dimethylaminopropyl]carbodiimide/N-hydroxysuccinimide EDC/NHS to form the primary amide such that the fluorescent dye is covalently linked to the triethoxysilane group by an 8 carbon linking unit, interrupted by a C(O)NH unit in the resulting modified IR-820-silane fluorescent dye, which was used without further purification to form the 3-7 nm NIR fluorescent nanoparticles.

All patents, patent applications, provisional applications, and publications referred to or cited herein are incorporated by reference in their entirety, including all figures and tables, to the extent they are not inconsistent with the explicit teachings of this specification.

1-33. (canceled)

34. A nanoparticle, comprising a metal oxide and a near-IR (NIR) fluorescent dye bound to the metal oxide wherein the nanoparticle has a diameter of about 3 nm to about 7,000 nm.

35. The nanoparticle of claim 34, wherein the diameter less than 8 nm.

36. The nanoparticle of claim 34, wherein a plurality of the nanoparticles is monodisperse.

37. The nanoparticle of claim 34, wherein the metal oxide is silicon oxide.

38. The nanoparticle of claim 34, wherein the NIR fluorescent dye is bound to the metal oxide by one or more covalent bonds.

39. The nanoparticle of claim 38, wherein the dye comprises a conjugated system from IR-27, IR-1048, IR-1061, IR-775, IR-780, IR-783, IR-797, IR-806, or IR-820.

40. The nanoparticle of claim 39, wherein the central carbon of the conjugated system is covalently bonded to a N, O, S, or C atom that is bound to the metal oxide through a series of 3 to 20 carbon-carbon bonds that is uninterrupted or interrupted by O, S, NH, NR, C(O)O, C(O)NH, or C(O)NR.

41. The nanoparticle of claim 34, wherein the NIR fluorescent dye is a naphthalocyanine or phthalocyanine metal complex.

42. The nanoparticle of claim 34, further comprising a metal deposition; at least one moiety that exhibits luminescence, magnetic properties, paramagnetic properties, or x-ray opacity; or any combination thereof.

43. The nanoparticle of claim 42, wherein the metal deposition is gold speckles.

44. The nanoparticle of claim 42, wherein the moiety that exhibits magnetic or paramagnetic properties is a transition metal chelate or a lanthanide chelate.

45. The nanoparticle of claim 44, wherein the transition metal chelate is Mn-EDTA.

46. The nanoparticle of claim 44, wherein the lanthanide chelate is Gd-DTPA.

47. The nanoparticle of claim 34, further comprising one or more habitat modifiers bound to the metal oxide through a series of 3 to 20 carbon-carbon bonds that is uninterrupted or interrupted by O, S, NH, NR, C(O)O, C(O)NH, or C(O)NR, wherein the habitat modifier is an organic or inorganic group that alters polarity, pH, dielectric permittivity and/or porosity within the metal oxide matrix of the nanoparticle.

48. The nanoparticle of claim 47, wherein the habitat modifier is derived from polyethylene glycol silane, dodecyl silane, ethylene glycol, or glycerin.

49. The nanoparticle of claim 34, further comprising one or more optical limiting moiety.

50. The nanoparticle of claim 49, wherein the optical limiting moiety comprises naphthalocyanine, phthalocyanine, fullerene, or functionalized fullerene.

51. The nanoparticle of claim 34, further comprising a temperature indicating agent and/or one or more chemotherapeutic agents, gene transfection agents, and/or gene silencing agents.

52. A method of forming a silica comprising nanoparticle according to claim 34, comprising:
providing at least one tetraalkoxysilane, an alcohol, water, and am ammonium catalyst; and
adding a polar aprotic solvent, wherein the silica nanoparticle formed has a diameter of about 3 to about 8 nm.

53. The method of claim 52, further comprising a fluorescent dye.

54. A method of in vivo and in vitro imaging, comprising:
administering to a target a NIR fluorescent dye comprising nanoparticle according to claim 34, wherein the nanoparticle is 3 to 50 nm diameter; and
detecting a fluorescence signal from the nanoparticle.

55. The method of claim 54, wherein the nanoparticle further comprises metal deposition; at least one moiety that exhibits luminescence, magnetic properties, paramagnetic properties, or x-ray opacity; or any combination thereof, wherein detecting further comprises one or more signals for photo acoustic tomography (PAT) imaging and at least one of luminescence imaging, magnetic resonance (MR) imaging and x-ray imaging.

56. The method of claim 54, wherein the nanoparticle further provides therapeutic active agents.
